(19) United States
(12) Patent Application Publication
(10) Pub. No.: US 2007/0128246 A1
(43) Pub. Date: Jun. 7, 2007

(54) Solventless method for forming a coating

(76) Inventors: Syed Faiyaz Ahmed Hossainy, Fremont, CA (US); Florian Ludwig, Mountain View, CA (US); Srinivasan Sridharan, Morgan Hill, CA (US); Stephen D. Pacetti, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/295,956

(22) Filed: Dec. 6, 2005

(51) Int. Cl. A61F 2/02; B05D 3/00

(52) U.S. Cl. 424/423; 427/2.24

(57) ABSTRACT

Provided herein are medical device having a coating substantially free from effects of drying kinetics and methods of forming the coating.

### BACKGROUND OF THE INVENTION

1. Field of the Invention

This invention generally relates to a medical device having a coating substantially free from effects of drying kinetics and methods of forming the coating.

2. Description of the Background

Percutaneous coronary intervention (PCI) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress against the atherosclerotic plaque of the lesion to remodel the lumen wall. The balloon is then deflated to a smaller profile to allow the catheter to be withdrawn from the patient's vasculature. In conjunction with balloon therapy, a stent can be used to prevent lumen recoil, to uphold the wall of the lumen, and to provide biological therapy.

A current paradigm in the art of PCI is to form a polymeric coating on the implant surface to modulate biological responses from the implant. In a coating process, a solvent is generally used to dissolve the coating polymer and/or a drug. The use of solvents, in particular volatile solvents, in polymer coatings results in drying kinetics after the surface deposition of the dissolved polymer. Drying kinetics occur rapidly. For example, when spray coating a stent, over 50% of the solvent evaporates in tens of seconds to just a few minutes. These drying kinetics are hard to control and make it difficult to predict process outcome. As a result, reproducibility of the same coating becomes difficult.

The embodiments of the present invention address these concerns as well as others that are apparent by one having ordinary skill in the art.

### SUMMARY OF THE INVENTION

Provided herein is a solvent free process for coating a medical device (e.g., a stent). The process includes coating the medical device with a solvent free coating formulation in which monomers of a coating polymer are used as the solvent. The process includes: (1) coating the solvent free coating formulation onto a medical device, and (2) curing via polymerization or crosslinking reactions to form a polymer coating. Where an agent is coated onto the medical device, e.g., a drug-delivery stent, the agent can be included in the solvent free coating formulation and/or coated onto the device in another layer, for instance, a neat drug layer on top of which the solvent free formulation can be coated as a topcoat. In some embodiments, the solvent free coating composition can be used to form a primer or a topcoat on the medical device if the drug includes a hydroxyl or amino group.

In some embodiments, the present invention provides a medical device having thereon a coating substantially free from effects of drying kinetics. The coating contains a polymeric material and optionally a bioactive agent and can be formed by the methods described herein.

The bioactive agent can be any bioactive agent known in the art. Some exemplary bioactive agents are paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, pimecrolimus, imatinib mesylate, midostaurin, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), clobetasol, prodrugs thereof, co-drugs thereof, and combinations thereof. The implantable device can be implanted in a patient to treat or prevent a disorder such as atherosclerosis, thrombosis, restenosis, hemorrhage, vascular dissection or perforation, vascular aneurysm, vulnerable plaque, chronic total occlusion, claudication anastomotic proliferation for vein and artificial grafts, bile duct obstruction, ureter obstruction, or tumor obstruction.

### DETAILED DESCRIPTION

Provided herein is a solvent free process for coating a medical device (e.g., a stent). The process includes coating the medical device with a solvent free coating formulation in which monomers of a coating polymer are used as the solvent. The process includes: (1) coating the solvent free coating formulation onto a medical device, and (2) curing via polymerization or crosslinking reactions to form a polymer coating. Where an agent is coated onto the medical device, e.g., a drug-delivery stent, the agent can be included in the solvent free coating formulation and/or coated onto the device in another layer, for instance, a neat drug layer on top of which the solvent free formulation can be coated as a topcoat. The neat drug layer can be produced by applying a drug/solvent composition to the device to form a drug layer free from a polymer or can be produced by applying a drug/solvent/polymer composition to the device to form a polymer layer having a drug. Formation of a primer layer on the surface of the device is also included within the scope of the embodiments of the present invention. For example, the solvent free coating composition can be used to form a primer or a topcoat on the medical device if the drug includes a hydroxyl or amino group.

In some embodiments, the present invention provides a medical device having thereon a coating substantially free from effects of drying kinetics. The coating contains a polymeric material and optionally a bioactive agent and can be formed by the methods described herein. As used herein, the term “effects of drying kinetics” refers to the multiple consequences of solvent evaporation, more specifically rapid solvent evaporation. These effects include sub-cooling the coating and causing ambient water to condense, having the drug and polymer components precipitate or phase separate in a non-equilibrium fashion, and/or a redistribution of drug in the coating from the rapid diffusion of solvent giving rise to chromatographic movement of the drug.

The bioactive agent can be any bioactive agent known in the art. Some exemplary bioactive agents are paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, pimecrolimus, imatinib mesylate, midostaurin, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), clobetasol, prodrugs thereof, co-drugs thereof, and combinations thereof. The implantable device can be implanted in a patient to treat or prevent a disorder such as atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, or tumor obstruction.

Crosslinking of Macromer Precursors

In some embodiments, the solvent free coating formulation includes a liquid macromer precursor of bio-rubber. In one embodiment, an active agent can be suspended in the coating formulation. The coating formulation can then be applied on a medical device and cured by, e.g., heat and/or catalysis, forming a bio-rubber coating that encapsulates the active agent in its solid phase. The macromer precursor of bio-rubber can be a macromer capable of crosslinking with a linking agent. In some embodiments, the macromer precursor is a polydiacid which can be crosslinked with a linking agent such as a diol or a hydrogen bonding agent. An example of the polydiacid is poly(sebacic acid-co-1,4-butanediol) with a linking agent, e.g., glycerol. Other examples of macromer precursors include, but are not limited to, poly(sebacic acid-co-poly(ethylene gycol)), poly(sebacic acid-co-poly(propylene glycol)), poly(sebacic acid-co-poly(tetramethylene glycol)), poly(sebacic acid-co-1,6-hexanediol), poly(sebacic acid-co-1,2-propanediol), poly(sebacic acid-co-1,3-propanediol), poly(adipic acid-co-poly(ethylene gycol)), poly(adipic acid-co-poly(propylene glycol)), poly(adipic acid-co-poly(tetramethylene glycol)), poly(adipic acid-co-1,6-hexanediol), poly(adipic acid-co-1,4-butanediol), poly(adipic acid-co-1,2-propanediol), poly(adipic acid-co-1,3-propanediol), poly(succinic acid-co-poly(ethylene gycol)), poly(succinic acid-co-poly(propylene glycol)), poly(succinic acid-co-poly(tetramethylene glycol)), poly(succinic acid-co-1,6-hexanediol), poly(succinic acid-co-1,4-butanediol), poly(succinic acid-co-1,2-propanediol), poly(succinic acid-co-1,3-propanediol), poly(lysine diisocyanate ethyl ester-co-poly(ethylene gycol)), poly(lysine diisocyanate ethyl ester-co-poly(propylene glycol)), poly(lysine diisocyanate ethyl ester-co-poly(tetramethylene glycol)), poly(lysine diisocyanate ethyl ester-co-1,6-hexandiol), poly(lysine diisocyanate ethyl ester-co-1,4-butanediol), poly(lysine diisocyanate ethyl ester-co-1,2-propanediol), poly(lysine diisocyanate ethyl ester-co-1,3-propanediol), poly(1,4-butane diisocyanate-co-1,6-hexanediol), poly(1,5-pentane diisocyanate-co-1,6-hexanediol), poly(1,6-hexane diisocyanate-co-1,6-hexanediol), and poly(1,2,7,8-diepoxyoctane-co-1,4-butanediamine) In some embodiments, for the macromer precursors to be flowable liquids at room temperature, the number average molecular weight should be less than about 20,000 Daltons. Crosslinking agents include, but are not limited to, diols, diamines, glycerol, pentaerythritol, trimethylol propane, poly(ethylene glycol) (PEG), poly(propylene glycol), poly(tetramethylene glycol), Jeffamines, glucose, fructose, saccharides, dithiols, such as dithiothreitol, and other molecules with two or more reactive groups, e.g., ethylene glycol, 1,2-propanediol, 1,3-propanediol, 1,4-butanediol, 1,5-pentanediol, 1,6-hexanediol, 1,7-heptanediol, 1,8-octanediol, 1,9-nonaediol, 1-10, decanediol, 1,11-undecanediol, 1,12-dodecane diol, C₂-C₁₂ branched or linear diols, C₂-C₁₂ branched or linear polyols, 1,4-cyclohexanediol, and cyclohexanedimethanol. By selecting the macromer and/or the crosslinking agents, coatings with various hydrophilicity/hydrophobicity and/or various biological properties (e.g., non-fouling) can be formed.

The crosslinking can be carried out under different conditions according to the nature of the crosslinking agent. A general guidance is that the crosslinking chemistry be compatible with the drug, if present, and not degrade the drug. One example of such crosslinking chemistry is using thiol terminated macromers which crosslink and undergo condensation polymerization via Michael addition. This crosslinking chemistry can be performed in vivo and can be non-toxic to cells. For example, a macromer precursor can be formed of methacrylates with α,β-unsaturated ester end groups by ATRP method (Coessens V., Pintauer T., Matyjaszewski K., Prog. Polym. Sci. 26 (2001) 337-377), which may undergo further polymerization by addition of dithiothreitol.

In some embodiments, the macromer precursors can be monomers and polymers of cyanoacrylates. These polymers can undergo anionic crosslinking in the presence of water so as to form a coating on the device. These macromer precursors include, but are not limited to, methyl 2-cyanoacrylate, ethyl 2-cyanoacrylate, n-propyl 2-cyanoacrylate, isopropyl 2-cyanoacrylate, n-butyl 2-cyanoacrylate, pentyl 2-cyanoacrylate, hexyl 2-cyanoacrylate, heptyl cyanoacrylate, octyl cyanoacrylate, and liquid oligomers of these cyanoacrylates.

In some embodiments, the macromer precursors can be silicone prepolymers. The crosslinking can be achieved by addition chemistries (e.g., addition of a Si—H grouping to a C═C bond) catalyzed by a catalyst (e.g., platinum colloids, platinum compounds, ruthenium compounds, iridium compounds, rhodium compounds, rhenium compounds, and/or combinations thereof) (Lee, Chi-Long, et al. U.S. Pat. No. 4,162,243). For example, a coating composition can be made to have a silicone and optionally a drug. The platinum catalyst can be added prior to coating to catalyze the crosslinking of silicone prepolymers to form silicone polymer, forming a solid coating (optionally with the drug in the coating).

In some other embodiments, the coating composition can include a polyurethane solvent-free formulation and a crosslinking agent with or without drug. Drugs with no hydroxyl or amino groups are compatible with polyurethane crosslinking chemistry. Crosslinking can be achieved using a crosslinking agent (or a linker) having two or more hydroxyl, amino and/or thiol functional groups to generate a solid coating. In some embodiments, the coating formulation can include an aliphatic polyurethane with a diol chain extender, and with or without a drug. Crosslinking can be achieved via one or two step polymerization between the polyurethane molecules to form a solid coating. In some embodiments, the coating formulation can include a polyurethane macromer with isocyanate groups, and a linker with hydroxy, amino, or thiol groups. Crosslinking can be achieved by single- or multiple-step polymerization between the polyurethane molecules via the linker.

Free Radical Curing

In some other embodiments, free radical chemistry can be used to cure the monomers. Free radicals can be generated by, e.g., ultraviolet light (UV) radiation or thermal initiation by heating with initiators or e-beam irradiation. For a UV curing process, free radical initiators can be those known in the art. Examples of UV free radical initiators include, but are not limited to, benzophenone, isopropyl thioxanone, 2,2-dimethoxy-2-phenyl-acetophenone (Nguyen K T, West J L, Biomaterials, 23 (2002) 4307-4314), 2-hydroxy-1-[4-(hydroxyethoxy)phenyl]-2-methyl-1-propanone, and Irgacure and Darocure photoinitiators (available from Ciba Specialty Chemicals, Tarrytown, N.Y.). Thermally activated free radical initiators include, but are not limited to, azobisisobutyronitrile, benzoyl peroxide, acetyl peroxide, lauryl peroxide, t-butyl peracetate, cumyl peroxide, t-butyl peroxide, and t-butyl hydroperoxide (Geroge Odian, Principles of Polymerization, 2nd Ed., John Wiley & Sons, 1981, New York). Different free radical initiators have different level of reactivity toward the drug molecule that may be present in the coating. For example, some initiators like benzophenone, when combined with acrylate monomers, create high concentrations of aggressive free radicals, which can react with the triene moiety of everolimus so as to degrade the drug. A less aggressive photoinitiator, such as isopropyl thioxanone and 2,2-dimethoxy-2-phenyl acetophenone, which photolyze with 365 nm light, are more suitable to use with drugs that are reactive to free radicals, which often have unsaturated carbon-carbon bonds. One of ordinary skill in the art can readily select a proper initiator for UV or thermal curing of a monomer in the coating process.

In some embodiments, the coating formulation can include poly(ortho ester) (POE) macromers such as diols and diketene acetals, and a drug. In another embodiment, the coating formulation can include a low reactivity monomer such as a methacrylate (e.g., butyl methacrylate), and a free radical initiator such as isopropyl thioxanone and 2,2-dimethoxy-2-phenyl acetophenone, and a drug. The coating formulation can be readily cured under UV to generate a drug-delivery coating.

In some embodiments, the coating formulation can include a low molecular weight poly(ethylene glycol) (PEG), e.g., PEG having a number average molecular weight (Mₙ) in the range between about 200 Daltons and about 300 Daltons, which is a liquid into which drug may be mixed and with which drug may be coated onto a medical device (e.g., stent) before it is crosslinked on the device. In some embodiments, the PEG can have polymerizable functional groups such as acrylate or methacrylate, via, e.g., an ester linkage. Crosslinking of the PEG can be readily achieved by adding a multifunctional agent (e.g., a multifunctional acrylate) into the coating formulation, followed by UV activation post-coating. Some other functional groups that can be attached to the PEG or another liquid polymer include, but are not limited to, groups that have at least one unsaturated carbon-carbon bond, such as methacrylates, fumarates, cinnamates, acroleins, and malonates and combinations thereof.

Other Methods

In some other embodiments, effects of drying kinetics in coating can be eliminated by coating macromers from a non-volatile solvent and crosslinking these macromers prior to (forced) solvent evaporation. This process can achieve the effects of the solvent-free process described above if the drug is not soluble in the non-volatile solvent in that the drying kinetics and processes will not effect the drug distribution or drug phase. As used herein, the term “non-volatile solvent” refers to a solvent having a low vapor pressure at ambient temperature. One example of such non-volatile solvent is water. Where water is used as solvent, the coating formulation may include hydrophilic macromers such as PVP (polyvinylpyrrolidone), PEG, PVA (poly(vinyl alcohol)), hyaluronic acid, poly(2-hydroxyethyl methacrylate) or other hydrophilic macromers that may be end-group functionalized, e.g., by acrylates and/or methacrylates with an initiator. The coating formulation can be coated onto a device (e.g., a stent) and cured by heat or UV. Alternatively, film-forming biopolymers such as albumin, collagen, gelatin, elastin etc., can be coated and then crosslinked on the surface of a device by formaldehyde, glutaraldehyde, carbodiimides such as EDC, bis-N-hydroxy succinimidyl ester derivatives, bis-vinyl sulfone derivatives, bis-N-maleimide derivatives, diisocyanates, UV light, dehydrothermal processing, and genipin. Alternatively, bifunctional, UV sensitive crosslinkers may be conjugated to functionalize macromers or biomacromers prior to coating the molecules onto the device such that the UV reactive functionality is available for crosslinking of the macromers on the device. For example, crosslinkers comprising a UV reactive crosslinking group and an N-hydroxysuccinimide (NHS) ester may be conjugated to amine groups present on the macromer prior to the coating, allowing to crosslink the modified macromers by UV radiation after coating them onto the device. Likewise, epoxide groups may be used to conjugate to amine groups present on the macromers, and maleimide or vinyl-sulfide groups may be used to conjugate the UV reactive crosslinker to thiol groups present on the macromers.

### Biocompatible Polymers and Biobeneficial Materials

In addition to the monomers, prepolymers or macromers previously described, the solvent free coating formulation can include any biocompatible polymer. Such biocompatible polymers can be any biocompatible polymer known in the art, which can be biodegradable or nondegradable. Biodegradable is intended to include bioabsorbable or bioerodable, unless otherwise specifically stated. Representative examples of polymers that can be used in accordance with the present invention include, but are not limited to, poly(ester amide), ethylene vinyl alcohol copolymer (commonly known by the generic name EVOH or by the trade name EVAL), poly(L-lactide), poly(D-lactide), poly(D,L-lactide), poly(D,L-lactide-co-L-lactide), poly(L-lactide-co-glycolide), poly(D,L-lactide-co-glycolide) (PLGA), poly(L-lactide-co-caprolactone), poly(D,L-lactide-co-caprolactone), poly(hydroxyvalerate), polycaprolactone, poly(hydroxybutyrate), poly(hydroxybutyrate-co-valerate), polydioxanone, polyorthoester, polyanhydride, poly(glycolic acid), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, fluoro polymers or copolymers under the trade name Solef™ or Kynar™ such as polyvinylidene fluoride (PVDF) and poly(vinylidene fluoride-co-hexafluoropropylene), polyvinylidene chloride, poly(butyl methacrylate), polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, isobutylene-styrene copolymers, methacrylate-styrene copolymer, ABS resins, and ethylene-vinyl acetate copolymers, polyamides, such as Nylon 66 and polycaprolactam, alkyd resins, polycarbonates, polyoxymethylenes, polyimides, polyethers, polyvinylpyrrolidone (PVP), poly(vinyl alcohol) (PVA), polyacrylamide (PAAm), poly(glyceryl sebacate), poly(propylene fumarate), epoxy resins, polyurethanes, rayon, rayon-triacetate, cellulose acetate, cellulose butyrate, cellulose acetate butyrate, cellophane, cellulose nitrate, cellulose propionate, cellulose ethers, and carboxymethyl cellulose.

The biocompatible polymer can provide a controlled release of a bioactive agent, if included in the coating and/or binding the bioactive agent to a substrate, which can be the surface of a medical device or a coating thereon. Controlled release and delivery of bioactive agent using a polymeric carrier has been extensively researched in the past several decades (see, for example, Mathiowitz, Ed., Encyclopedia of Controlled Drug Delivery, C.H.I.P.S., 1999). For example, PLA based drug delivery systems have provided controlled release of many therapeutic drugs with various degrees of success (see, for example, U.S. Pat. No. 5,581,387 to Labrie, et al.). The release rate of the bioactive agent can be controlled by, for example, by selection of a particular type of biocompatible polymer which can provide a desired release profile of the bioactive agent. The release profile of the bioactive agent can be further controlled by the molecular weight of the biocompatible polymer and/or the ratio of the biocompatible polymer over the bioactive agent. In the case of a biodegradable polymer, the release profile can also be controlled by the degradation rate of the biodegradable polymer. One of ordinary skill in the art can readily select a carrier system using a biocompatible polymer to provide a controlled release of the bioactive agent.

A preferred biocompatible polymer is a polyester, such as one of poly(ester amide), poly(D,L-lactide) (PDLL), poly(D,L-lactide-co-glycolide) (PLGA), polyglycolic acid (PGA), poly(glycolide), polyhydroxyalkanoate (PHA), poly(3-hydroxybutyrate) (PHB), poly(3-hydroxybutyrate-co-3-hydroxyvalerate), poly((3-hydroxyvalerate), poly(3-hydroxyhexanoate), poly(4-hydroxybutyrate), poly(4-hydroxyvalerate), poly(4-hydroxyhexanoate), poly(D,L-lactic acid), poly(L-lactide), poly(L-lactide-co-D,L-lactide), polycaprolactone (PCL) and a combination thereof.

In some embodiments, the solvent free coating formulation can include a biobeneficial material. The biobeneficial material can be a polymeric material or non-polymeric material. The biobeneficial material is preferably flexible when present as a discrete layer, or confers elastic properties in a blend or copolymer, and is biocompatible and/or biodegradable, more preferably non-toxic, non-antigenic and non-immunogenic. A biobeneficial material is one which enhances the biocompatibility of a device by being non-fouling, hemocompatible, actively non-thrombogenic, or anti-inflammatory, all without depending on the release of a pharmaceutically active agent. As used herein, the term non-fouling is defined as preventing, delaying or reducing the amount of formation of protein build-up caused by the body's reaction to foreign material and can be used interchangeably with the term “anti-fouling.”

Representative biobeneficial materials include, but are not limited to, polyethers such as poly(ethylene glycol), copoly(ether-esters) (e.g. PEO/PLA); polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropyl methacrylamide, PEG acrylate (PEGA), PEG methacrylate, 2-methacryloyloxyethylphosphorylcholine (MPC) and poly(n-vinyl pyrrolidone) (PVP), polymers containing carboxylic acid bearing monomers such as methacrylic acid (MA), acrylic acid (AA), alkoxymethacrylate, alkoxyacrylate, and 3-trimethylsilylpropyl methacrylate (TMSPMA), polystyrene-polyisoprene-polystyrene-co-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, collagen, dextran, dextrin, hyaluronic acid, fragments and derivatives of hyaluronic acid, heparin, fragments and derivatives of heparin, glycosamino glycan (GAG), GAG derivatives, polysaccharide, elastin, chitosan, alginate, silicones, and combinations thereof.

In some embodiments, the biocompatible polymer or biobeneficial material can exclude any one of the aforementioned materials.

In some embodiments, the biobeneficial material is a block copolymer comprising flexible poly(ethylene glycol terephthalate)/poly(butylene terephthalate) (PEGT/PBT) segments (PolyActive™). These segments are biocompatible, non-toxic, non-antigenic and non-immunogenic. Previous studies have shown that the PolyActive™ top coat decreases the thrombosis and embolism formation on stents. PolyActive™ is generally expressed in the form of xPEGTyPBTz, in which x is the molecular weight of PEG, y is percentage of PEGT, and z is the percentage of PBT. A specific PolyActive™ polymer can have various ratios of the PEG, ranging from about 1% to about 99%, e.g., about 10% to about 90%, about 20% to about 80%, about 30% to about 70%, about 40% to about 60% PEG. The PEG for forming PolyActive™ can have a molecular weight ranging from about 300 Daltons to about 100,000 Daltons, e.g., about 300 Daltons, about 500 Daltons, about 1,000 Daltons, about 5,000 Daltons, about 10,000 Daltons, about 20,000 Daltons, or about 50,000 Daltons.

In some embodiments, the biobeneficial material can be a polyether such as PEG or polyalkylene oxide.

### Bioactive Agents

The bioactive agents can be any diagnostic, preventive and therapeutic agents. Examples of such agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Other examples of drugs include antibodies, receptor ligands, and enzymes, adhesion peptides, oligosaccharides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Such agents can also include a prohealing drug that imparts a benign neointimal response characterized by controlled proliferation of smooth muscle cells and controlled deposition of extracellular matrix with complete luminal coverage by phenotypically functional (similar to uninjured, healthy intima) and morphologically normal (similar to uninjured, healthy intima) endothelial cells. Such agents can also fall under the genus of antineoplastic, cytostatic, anti-inflammatory, antiplatelet, anticoagulant, antifibrin, antithrombin, antimitotic, antibiotic, antiallergic and antioxidant substances. Examples of such antineoplastics and/or antimitotics include paclitaxel (e.g. TAXOL® by Bristol-Myers Squibb Co., Stamford, Conn.), docetaxel (e.g. Taxotere®, from Aventis S.A., Frankfurt, Germany) methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include heparinoids, hirudin, recombinant hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist, antibody, and thrombin inhibitors such as Angiomax a (Biogen, Inc., Cambridge, Mass.). Examples of cytostatic agents include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.), actinomycin D, or derivatives and analogs thereof (manufactured by Sigma-Aldrich 1001 West Saint Paul Avenue, Milwaukee, Wis. 53233; or COSMEGEN available from Merck). Synonyms of actinomycin D include dactinomycin, actinomycin IV, actinomycin I₁, actinomycin X₁, and actinomycin C₁. Other drugs include calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium.

Other therapeutic substances or agents which may be appropriate include alpha-interferon, genetically engineered epithelial cells, bioactive RGD, antibodies such as CD-34 antibody, abciximab (REOPRO), and progenitor cell capturing antibody, prohealing drugs that promotes controlled proliferation of muscle cells with a normal and physiologically benign composition and synthesis products, enzymes, antivirals, anticancer drugs, anticoagulant agents, free radical scavengers, steroidal anti-inflammatory agents, glucocorticoids, non-steroidal anti-inflammatory agents, antibiotics, nitric oxide donors, super oxide dismutases, super oxide dismutases mimics, 4-amino-2,2,6,6-tetramethylpiperidine-1-oxyl (4-amino-TEMPO), dexamethasone, clobetasol, aspirin, estradiol, tacrolimus, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, 40-O-tetrazole-rapamycin, 40-epi-(N-1-tetrazolyl)-rapamycin (ABT-578), pimecrolimus, imatinib mesylate, midostaurin, progenitor cell capturing antibody, pro-drugs thereof, co-drugs thereof, and a combination thereof. The foregoing substances are listed by way of example and are not meant to be limiting. Other active agents which are currently available or that may be developed in the future are equally applicable.

### Examples of Medical Device

As used herein, a medical device may be any suitable medical substrate that can be implanted in a human or veterinary patient. Examples of such medical devices include self-expandable stents, balloon-expandable stents, stent-grafts, grafts (e.g., aortic grafts), artificial heart valves, cerebrospinal fluid shunts, pacemaker electrodes, and endocardial leads (e.g., FINELINE and ENDOTAK, available from Guidant Corporation, Santa Clara, Calif.). The underlying structures can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention. For example, the device can be a bioabsorbable stent, made from a polymeric material (and/or an erodable metal). The bioabsorbable stent can include a drug coating, for example with a polymer film layer or the drug can be compounded or embedded in the body of the stent.

### Method of Use

A medical device (e.g., stent) having any of the above-described features is useful for a variety of medical procedures, including, by way of example, treatment of obstructions caused by tumors in bile ducts, esophagus, trachea/bronchi and other biological passageways. A stent having the above-described coating is particularly useful for treating occluded regions of blood vessels caused by abnormal or inappropriate migration and proliferation of smooth muscle cells, thrombosis, restenosis, and vulnerable plaque. Stents may be placed in a wide array of blood vessels, both arteries and veins. Representative examples of sites include the iliac, renal, and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter which allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent with or without a drug delivery coating may then be expanded at the desired area of treatment. A post-insertion angiogram may also be utilized to confirm appropriate positioning.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method of forming a coating on a medical device, comprising:
(a) providing a solvent free liquid coating formulation,
(b) coating the formulation on at least an area of the medical device to form a layer of liquid coating, and
(c) curing the layer of liquid coating to generate a solid coating.

2. The method of claim 1, wherein the liquid coating formulation comprises a drug.

3. The method of claim 1, wherein the liquid coating formulation comprises a prepolymer and a drug.

4. The method of claim 1, wherein the liquid coating formulation comprises a prepolymer.

5. The method of claim 1, wherein the solvent free liquid coating formulation includes a free radical polymerizable monomer or polymer and a free radical initiator, and
wherein the curing comprises curing by ultraviolet light radiation, thermal initiation, or e-beam curing.

6. The method of claim 1, wherein the solvent free liquid coating formulation comprises a diacid polymer or prepolymer, and a crosslinking agent that includes at least two functional groups selected from the groups consisting of hydroxyls, amino groups, carboxyls, thiols, isocyanates, isothiocyanates, acid chlorides, epoxides and combinations thereof.

7. The method of claim 1, wherein the solvent free liquid coating formulation includes a prepolymer formed of monomers having α,β-unsaturated ester or amide groups and an agent having at least two functional groups selected from the groups consisting of hydroxyls, amino groups, thiols, and combinations thereof.

8. The method of claim 7, wherein the monomers are methacrylates, acrylates, or combinations thereof, and
wherein the agent is dithiothreitol.

9. The method of claim 1, wherein the solvent free coating formulation comprises:
a poly(ortho ester) liquid macromer selected from the group consisting of a diol, a diketene acetal, and combinations thereof, and
optionally a drug.

10. The method of claim 1, wherein the solvent free coating formulation comprises a photoinitiator, a methacrylate monomer, and optionally a drug.

11. The method of claim 1, wherein the solvent free coating formulation comprises a polyurethane macromer with isocyanate groups, a linker with hydroxy, amino, or thiol groups, and optionally a drug.

12. The method of claim 1, wherein the solvent free coating formulation comprises a polyurethane macromer with hydroxy, amino, or thiol groups, a linker with diisocyanate or diisothiocyanate groups, and optionally a drug.

13. The method of claim 1, wherein the solvent free coating formulation comprises a diepoxide macromer, a linker with hydroxy, amino or thiol groups, and optionally a drug.

14. The method of claim 14, wherein the solvent free coating formulation comprises a low molecular weight poly(ethylene glycol) (PEG).

15. The method of claim 14, wherein the PEG has a number average molecular weight from about 200 Daltons to about 300 Daltons.

16. The method of claim 15, wherein the PEG comprises acrylate or methacrylate functional groups, and wherein the solvent free coating formulation further comprises a crosslinking agent having at least two acrylate or methacrylate functional groups.

17. The method of claim 1, wherein the solvent free coating formulation comprises a polymer having a UV curing group that has a carbon-carbon unsaturated bond.

18. The method of claim 17, wherein the UV curing group is selected from the group consisting of acrylates, methacrylates, fumarates, cinnamates, acrolein, malonates, and combinations thereof.

19. The method of claim 1, wherein the solvent free coating formulation comprises water and a macromer which comprises an alkyl 2-cyanoacrylate.

20. The method of claim 1, wherein solvent free coating formulation comprises silicone prepolymers and a catalyst, whereby the catalyst catalyzes polymerization of the silicone prepolymers so as to allow the post-coating formation of a solid coating.

21. The method of claim 20, wherein the catalyst is a platinum catalyst.

22. The method of claim 1, wherein medical device includes a drug and wherein the solvent free coating formulation is a primer or a topcoat on the medical device if the drug used includes a hydroxyl or amino group.

23. The method of claim 1, wherein the solvent free coating formulation includes an aliphatic polyurethane and optionally a drug, wherein the polyurethane comprises a diol chain extender.

24. The method of claim 1, wherein the liquid coating formulation is coated on a layer including a drug.

25. A method of forming a coating substantially free of effects of drying kinetics, comprising
(a) providing a coating formulation that comprises a macromer having at least one functional group, a non-volatile solvent, and a drug,
(b) coating the formulation on a medical device to form a layer of liquid coating,
(c) curing the layer of liquid coating prior to the evaporation of the solvent, and
(d) allowing the non-volatile solvent to evaporate so as to form a solid coating,
wherein the drug is not soluble in the non-volatile solvent.

26. The method of claim 25, wherein the non-volatile solvent is water, and wherein the macromer is a hydrophilic polymer.

27. The method of claim 25, wherein the macromer is a polymer selected from the group consisting of poly(vinylpyrrolidone) (PVP), PEG, PVA, hyaluronic acid, poly(2-hydroxyethyl methacrylate), and combinations thereof.

28. A method of forming a coating substantially free of effects of drying kinetics, comprising
(a) providing a coating formulation that comprises a film-forming biopolymer, a non-volatile solvent, and optionally a drug,
(b) coating the formulation on a medical device to form a layer of liquid coating,
(c) curing the layer of liquid coating prior to the evaporation of the solvent, and
(d) allowing the non-volatile solvent to evaporate so as to form a solid coating,
wherein the drug is not soluble in the non-volatile solvent.

29. The method of claim 26, wherein the non-volatile solvent is water, and
wherein the biopolymer is hyaluronic acid, albumin, gelatin, collagen, chondroitan sulfate, chitosan, heparin, and combinations thereof.

30. The method of claim 1, wherein the medical device is a stent.

31. The method of claim 25, wherein the medical device is a stent.

32. The method of claim 28, wherein the medical device is a stent.

33. A medical device comprising a substrate and a coating on the substrate, wherein the coating is substantially free from effects of drying kinetics, and wherein the coating comprises a polymeric material and optionally a bioactive agent.

34. The medical device of claim 33, wherein the coating is formed by the method of claim 1.

35. The medical device of claim 33, wherein the coating is formed by the method of claim 25.

36. The medical device of claim 33, wherein the coating is formed by the method of claim 28.

37. The medical device of claim 33, wherein the bioactive agent is selected from the group consisting of paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), clobetasol, pimecrolimus, imatinib mesylate, midostaurin, prodrugs thereof, co-drugs thereof, and a combination thereof.

38. A method of treating, preventing or ameliorating a disorder in a patient comprising implanting in the patient the implantable device of claim 37,
wherein the disorder is selected from atherosclerosis, thrombosis, restenosis, hemorrhage, vascular dissection or perforation, vascular aneurysm, vulnerable plaque, chronic total occlusion, patent foramen ovale, claudication, anastomotic proliferation for vein and artificial grafts, bile duct obstruction, ureter obstruction, or tumor obstruction.
